(19) United States
(12) Patent Application Publication
(10) Pub. No.: US 2004/0059317 A1
(43) Pub. Date: Mar. 25, 2004

(54) Drilling tool for a surgical drill

(75) Inventor: Reiner Hermann, Fridingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/606,950

(22) Filed: Jun. 18, 2003

Related U.S. Application Data
(63) Continuation of application No. PCT/EP01/14424, filed on Dec. 7, 2001.

(30) Foreign Application Priority Data
Dec. 23, 2000 (DE) 100 64 975

(51) Int. Cl. A61B 17/00

(52) U.S. Cl. 606/1

(57) ABSTRACT

In order to provide effective protection in the vicinity of a drill bit in a simple manner in the case of a drilling tool for a surgical drilling machine including a drill bit which comprises a shaft, a tip and a coupling member for establishing a rotary connection to a rotary drive for the drilling machine, it is proposed that a surrounding proximal protective sleeve having a proximal and a distal end be mounted on the shaft and a distal protective sleeve be resiliently insertable into said proximal protective sleeve and surround the drill bit between the distal end and the tip of the drill bit over at least a portion of the length thereof.

The present disclosure relates to the subject matter disclosed in international application PCT/EP 01/14424 of Dec. 7, 2001, which is incorporated herein by reference in its entity and for all purposes.

### BACKGROUND OF THE INVENTION

The invention relates to a drilling tool for a surgical drilling machine including a drill bit which comprises a shaft, a tip and a coupling member for establishing a rotary connection to a rotary drive for the drill.

Drill bits of this type can be constructed in a very large variety of ways and here, it could be a conventional spiral drill, an end-milling cutter, a hole boring drill, a K-wire (spike-wire) or other forms of tool which are fed into bone by means of a rotary movement.

When using drilling tools of this type on the human body, there is always a danger that the surrounding tissue could be injured by the rotating drill bit.

For this purpose, it is already known to surround the drill bit with a protective sleeve which can be inserted into the drilling machine in a resilient manner (PCT/AU96/00833). However, this form of construction leads to the drilling machine having to be constructed in a very much more costly manner because space surrounding the motor has to be available therein for accommodating the protective sleeve. Moreover, it is not possible to match the dimensions of the protective sleeve to the dimensions of the currently used drill bit in this manner so that, on the whole, this form of construction has not proved successful.

The object of the invention is to produce a drilling tool of the type mentioned in the first part of the main Claim which will not give rise to the fear of injury to the surrounding tissue and which can be employed in conventional drilling machines.

### SUMMARY OF THE INVENTION

In accordance with the invention, this object is achieved in the case of a drilling tool of the type described hereinabove in that a surrounding proximal protective sleeve having a proximal and a distal end is mounted on the shaft of the drill bit and a distal protective sleeve is resiliently insertable into said proximal protective sleeve such as to surround the drill bit between the distal end and the tip of the drill bit over at least a portion of the length thereof.

Thus, a two-part protective device is arranged directly on the shaft of the drill bit, this two-part protective device comprising a proximal protective sleeve that is itself fixed to the shaft and a distal protective sleeve which is resiliently insertable therein. Together, the drill bit and this two-part protective device form a drilling tool which can be inserted into a drilling machine in the conventional manner.

Hereby, the protective device is in each case matched to the size of the drill bit since the protective device is envisaged for a specific drill bit and consequently special matching of the drilling machine thereto is not necessary. In particular, such a drilling tool can be inserted into a drilling machine single-handedly in the conventional manner and, in addition, the drilling machine can be manipulated single-handedly, the drill bit thereby being completely masked externally when in use and, when the drill bit enters the material being treated, the distal protective sleeve will be displaced in such a manner that the drilling process itself can also be effected single-handedly.

It is expedient if the distal protective sleeve masks the drill bit to beyond the tip thereof when in the extended state so that there will then be no uncovered area of the drill bit which could cause injuries.

It is expedient if the proximal protective sleeve is mounted on the shaft of the drill bit such as to be rotatable about the longitudinal axis thereof. The drill bit can then rotate with respect to the proximal protective sleeve so that the rotational movement of the drill bit will not be transmitted directly to the proximal protective sleeve.

It is especially advantageous if the proximal protective sleeve and the shaft of the drill bit comprise mutually facing open peripheral grooves in which there engages at least one common bearing element. This results in the two-parts being mutually fixed in the axial direction whilst still maintaining a rotational coupling therebetween.

In particular, the bearing element may be elastically deformable and snap resiliently into one of the two peripheral grooves when the proximal protective sleeve is displaced axially relative to the shaft. The proximal protective sleeve and the shaft can thereby be coupled in the simplest of manners by means of a mutual displacement in the axial direction; as soon as the two peripheral grooves are mutually superimposed, the resilient bearing element snaps into the two grooves and fixes the two parts relative to one another, although this coupling is still capable of being released by the action of overcoming a certain axial force so that the entire protective device can easily be withdrawn from the drill bit.

It is advantageous hereby, if one peripheral groove is very shallow whilst the depth of the other peripheral groove is greater. The peripheral groove of greater depth thereby permanently accommodates the bearing element which is preferably in the form of a ring, and this bearing element then snaps into the peripheral groove of lesser depth in order to form a releasable axial fixing arrangement.

In a preferred embodiment, provision is made for the proximal protective sleeve to comprise a rotation preventing means which will prevent the proximal protective sleeve from rotating relative to the drilling machine when a drill bit is inserted in the drilling machine. It is thereby ensured that the proximal protective sleeve will remain non-rotatable relative to the drilling machine when this is in use, i.e. no rotational movement of the drill bit whatsoever will be transferred to the proximal protective sleeve, this thereby providing maximum safety since the stationary proximal protective sleeve cannot damage the surrounding tissue by dragging it along with it as a result of the rotational movement thereof.

For example, the rotation preventing means may be formed by a radial projection of the proximal protective sleeve which engages in a recess in the drilling machine.

It is also advantageous if the proximal protective sleeve is adapted to be fixed in the drilling machine in the axial direction, this thereby ensuring that, when in use, the proximal protective sleeve cannot be displaced by the drill bit.

This fixing function may, for example, be effected in that the proximal protective sleeve comprises at least one recess into which a locking projection of the drilling machine is insertable.

In a particularly preferred embodiment, provision is made for the proximal protective sleeve to carry a stop member with the aid of which it prevents displacement of the drill bit in the distal direction. Consequently, the proximal protective sleeve can additionally take over the role of axially fixing the drill bit in the drilling machine in that when the proximal protective sleeve is fixed in the drilling machine in the axial direction in the manner described, it will prevent axial displacement of the drill bit from the drilling machine by means of this stop member.

It is advantageous hereby if the coupling member engages axially in a coupling seating in the drilling machine in form-locking manner. This coupling is then maintained by virtue of the fact that axial displacement of the drill bit relative to the drilling machine is prevented, in the case of the example described, by the axial fixing of the proximal protective sleeve and the stop member of the protective sleeve on the drill bit.

In a particularly preferred embodiment of the invention, provision is made for the shaft of the drill bit to be provided with a collar which has an edge that adjoins a step of the proximal protective sleeve and comprises a peripheral groove for accommodating an O-ring, wherein said O-ring engages in an opposed peripheral groove in the proximal protective sleeve, the end face of said collar being provided with a driver means which is insertable in the form of a coupling member into a driver opening in the rotary drive for the drilling machine. Hereby, the collar may be formed in one piece with the shaft, it also being possible for the collar to be a part of synthetic material which is applied to the shaft of the drill bit and fixed thereon, for example, by using an extrusion-coating process around the shaft of the drill bit.

Furthermore, it is advantageous if the shaft is surrounded by a helical spring which is supported at one end on the shaft and on the distal protective sleeve at the other.

Hereby, it is expedient if, at at least one end thereof, the helical spring comprises an end winding which extends transversely relative to the longitudinal axis thereof and serves to support the spring on a support surface of the shaft or the distal protective sleeve. Rotation of the helical spring relative to the support surfaces is thereby possible, namely, in both directions should this be necessary.

In a special embodiment, the helical spring could also be coupled to the proximal protective sleeve in non-rotational manner, for example, by means of an injection moulding process into a part of the drill bit formed of synthetic material.

It is expedient if the distal protective sleeve is not rotatable with respect to the proximal protective sleeve about the longitudinal axis thereof. The two protective sleeves thus form a unit that is only rotatable in common, so that, if the proximal protective sleeve is prevented from rotation relative to the drilling machine, then this also applies to the distal protective sleeve. Consequently, in this arrangement, the drill bit is surrounded by a protective device which does not revolve with the drill bit, this thus providing maximum safety in the vicinity of the drill bit.

In order to achieve this effect, provision may be made for example, for the distal protective sleeve to be of non-round cross-section and to dip into a complementary opening in the proximal protective sleeve. The non-round cross-section may, for example, be produced by flattening a distal protective sleeve of circular cross-section on at least one side thereof.

It is expedient if the distal protective sleeve carries a depth scale so that the operator is then able to determine with the aid of this depth scale as to how far the distal protective sleeve has been inserted into the proximal protective sleeve, this being a measure for the amount by which the drill bit has advanced into the material being treated since the distal protective sleeve is supported on the surface of this material.

In a particularly preferred embodiment, there is provided a slip ring which is arranged on the distal protective sleeve and is retained thereon by friction, said slip ring being displaceable in the axial direction by overcoming the frictional force. When the distal protective sleeve dips into the proximal protective sleeve, this slip ring will be displaced and will indicate, following the resilient outward movement of the distal protective sleeve from the proximal protective sleeve, the maximally extent to which the distal protective sleeve has dipped into the proximal protective sleeve, the drilling depth achieved being readable thereon.

The following description of preferred embodiments of the invention will serve, in conjunction with the drawing, for a more detailed explanation.

### BRIEF DESCRIPTION OF THE DRAWINGS

FIG. 1 shows a longitudinal sectional view through a drilling tool incorporating a proximal and a distal protective sleeve when using a drill bit in the form of a spiral drill bit;

FIG. 2 a view similar to that of FIG. 1 when using a drill bit in the form of a K-wire in the case of a drill bit that has been advanced into the material being drilled;

FIG. 3 a sectional view along the line 3-3 in FIG. 1 and

FIG. 4 an enlarged detailed view of the area A in FIG. 1.

### DETAILED DESCRIPTION OF THE INVENTION

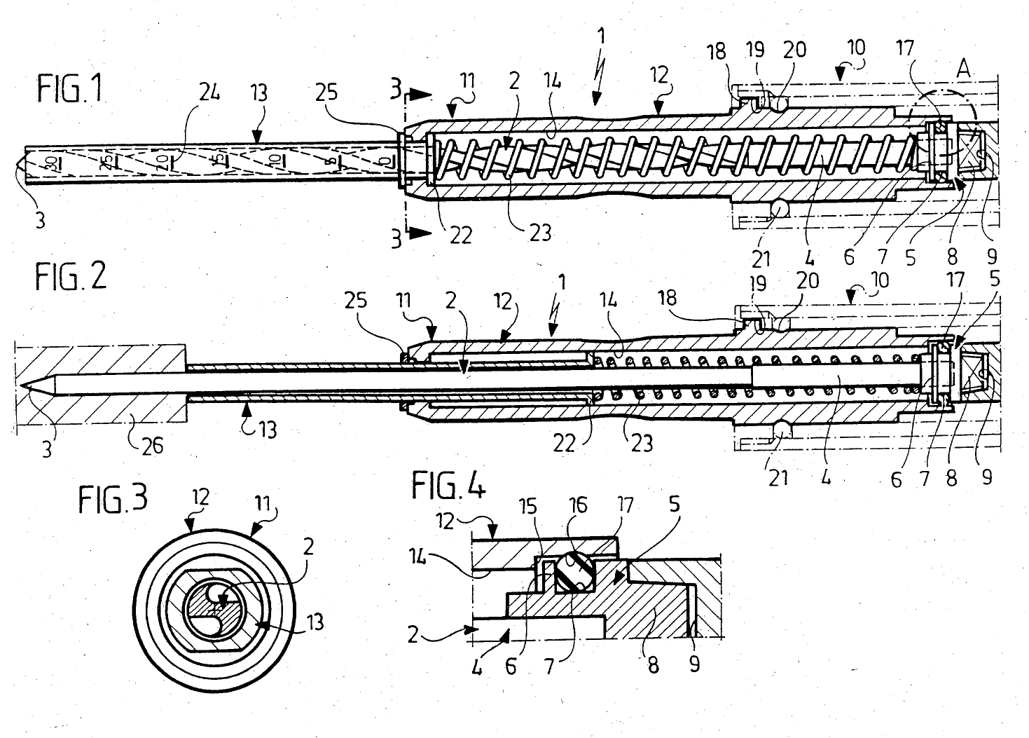

The drilling tool 1 illustrated in the drawing comprises a drill bit 2 in the form of a spiral drill in the embodiment of FIG. 1, and in the form of a K-wire in the embodiment of FIG. 2, the embodiments shown in FIGS. 1 and 2 otherwise being similar.

A shaft 4 supporting a collar 5 of enlarged outer diameter at the proximal end thereof adjoins the tip 3 of the drill bit 2 at the distal end thereof in the proximal direction. This collar may be formed in one piece with the drill bit 2 or it may consist of a separate component which is rigidly connected to the shaft 4. For example, the collar 5 may consist of a synthetic material and be attached to the proximal end of the shaft 4 by means of an extrusion-coating process.

This collar 5 surrounds the proximal end of the shaft 4 in sleeve-like manner and forms an annular step 6 which is produced by enlarging the periphery of the collar 5. A peripheral groove 7, into which there is laid a resilient O-ring 17 that projects outwardly by a very small amount beyond the contour of the peripheral groove 7, is worked into the periphery of the collar 5 in the immediate vicinity of this annular step 6.

At the end face thereof, the collar 5 is provided with a diametrically extending drive member 8 which projects in the proximal direction and forms a coupling member that is insertable into a coupling seating 9 in snug-fitting manner, said coupling seating being located in a drilling machine 10 that is only indicated by dash-dotted lines in the drawing and being driven in rotary manner about the longitudinal axis of the drill bit 2 by a rotary drive means that is not illustrated in the drawing.

The drill bit 2 is surrounded by a two-part protective housing 11 which comprises a proximal protective sleeve 12 and a distal protective sleeve 13.

The proximal protective sleeve 12 is of substantially circular cylindrical shape and surrounds the shaft 4 of the drill bit 2, but it is spaced therefrom so that an annular space 14 is formed between the shaft 4 and the inner wall of the proximal protective sleeve 12.

This annular space 14 is widened at the proximal end thereof into a step 15, the diameter of the annular space 14 thereby being less than the outer diameter of the annular step 6 of the collar 5 at the distal side of the step 15 but being a little larger at the proximal side of the step 15.

On the inner surface of the annular space 14 at the proximal side of the step 15, there is a shallow peripheral groove 16 having a cross-section which is in the form of an arc of a circle in the embodiment illustrated. When the proximal protective sleeve 12 is pushed axially onto the drill bit 2, the O-ring 17 located in the peripheral groove 7 snaps into the peripheral groove 16 so that the proximal protective sleeve 12 is thereby fixed relative to the drill bit 2 in the axial direction. This fixing arrangement can be released by overcoming a certain axial force so that it is easily possible to withdraw the drill bit 2 from the proximal protective sleeve 12 should this be desired.

When the peripheral groove 16 is located opposite the peripheral groove 7, the step 15 is then closely adjacent to the annular step 6 and hence this step 15 will then form a stop member with the aid of which the drill bit 2 will be moved in the same direction when the proximal protective sleeve 12 is inserted axially into the drilling machine 10 so that the drive member 8 can enter the coupling seating 9.

On the outer surface thereof, the proximal protective sleeve 12 is provided with a radially projecting pin 18 which enters an axial groove 19 in the drilling machine 10 when the proximal protective sleeve 12 is inserted into the drilling machine 10, this thereby preventing rotation of the proximal protective sleeve 12 in the drilling machine 10 about the longitudinal direction thereof.

Furthermore, on the outer surface thereof, the proximal protective sleeve 12 comprises a shallow peripheral groove 20 having a cross-section in the form of an arc of a circle into which a ball-shaped locking body 21 can enter, said body being mounted in a corresponding guide means in the drilling machine 10 such that it is displaceable in the radial direction. When inserted in the peripheral groove 20, this locking body 21 fixes the proximal protective sleeve 12 in the drilling machine 12 in the axial direction, whereas, when the locking body 21 is pushed back, the proximal protective sleeve 12 is then released and can thus be withdrawn from the drilling machine 10 in the axial direction.

The proximal protective sleeve extends over approximately half the length of the drill bit 2. The distal protective sleeve 13 is insertable in telescopic manner into the proximal protective sleeve 12, said distal protective sleeve surrounding the distal part of the shaft 4 of the drill bit 2 and extending up to the tip 3 of the drill bit 2 when fully extended from the annular space 14, as is illustrated in FIG. 1. A collar-like enlarged section 22 prevents the distal protective sleeve 13 from being withdrawn completely from the proximal protective sleeve 12.

A helical spring 23 that surrounds the proximal part of the shaft 4 of the drill bit 2 and is located in the annular space 14 is supported upon this collar-like enlarged section 22, the other end of the helical spring 23 being supported on the collar 5 whereat a rigid connection between the collar 5 and the helical spring 23 may exist, for example, the proximal end of the helical spring 23 may be injection moulded into the collar 5 if this consists of an injection moulding of synthetic material.

The distal protective sleeve 13 is normally extended in its entirety from the proximal protective sleeve 12 by means of this helical spring 23, but the helical spring 23 will be compressed when the distal protective sleeve 13 is inserted into the proximal protective sleeve 12.

The distal protective sleeve 13 is of circular cross-section but is flattened on opposite sides thereof as is clearly apparent from FIG. 3. The annular space 14 is provided with a substantially complementary opening at the distal end of the proximal protective sleeve 12 so that the distal protective sleeve 13 cannot rotate relative to the proximal protective sleeve 12 about the longitudinal direction thereof, but nevertheless it is displaceable in the longitudinal direction.

A depth scale 24 is inscribed on the distal protective sleeve 13, and, moreover, a ring 25 is pushed onto the distal protective sleeve 13, said ring being retained on the distal protective sleeve 13 by friction but being displaceable along the distal protective sleeve 13 by overcoming a certain fictional force.

The drill bit 2 and the two protective sleeves 12 and 13 surrounding it together form the drill tool 1 which, as a constructional entity, is then manipulable in a manner similar to that of a conventional drill. This constructional unit can easily be connected to the drilling machine by hand in that the constructional unit is inserted into the drilling machine 10 in the axial direction until the drive member 8 engages in the coupling seating 9. It is thereby ensured that the radially projecting pin of the proximal protective sleeve 12 enters the axial groove 19 so that the protective housing 11 is connected to the drilling machine 10 in non-rotatable manner. Axial fixing of the protective housing 11 and hence too of the drill bit 2 is obtained by the entry of the locking body 21 into the peripheral groove 20.

When beginning drilling, the drill bit 2 is completely surrounded over its entire length by the protective housing 11. During the drilling process, the tip 3 of the drill bit 2 penetrates the material 26 being treated (FIG. 2), the distal protective sleeve 13 thereby being supported on this material 26 and, in turn, being pushed into the proximal protective sleeve 12 against the effect of the helical spring 23 whilst the drill bit is being inserted into the material 26. The part of the drill bit 2 located outside the material 26 thereby remains permanently screened so that rotating parts cannot come into contact with the surrounding tissue.

For cleaning purposes, the drill bit 2 and the protective housing 11 can easily be separated from one another, it suffices to withdraw the drill bit backwardly out of the protective housing 11 and then the two parts can immediately be separated in the axial direction as soon as the O-ring 17 snaps out of the peripheral groove 16. It is also possible in this manner to utilise a particular protective housing 11 with different drill bits 2.

The depth to which the drill bit 2 is inserted can be read off the depth scale 24, the ring 25 being displaced along the depth scale 24 on the distal protective housing sleeve 12 by the proximal protective sleeve when the distal protective sleeve 13 is inserted into the proximal protective sleeve 12 whereby the ring will remain in the position of greatest displacement so that the maximum depth of entry can be read-off therefrom.

The protective housing 11 could also consist of various materials, metallic materials may be used but sterilisable synthetic materials could also be considered.

If the collar 5 consists of a synthetic material, the connection with the shaft of the drill bit 2 can be produced in the most varied of manners, the one explained above involving the extrusion-coating of the shaft, however it is also possible to make use of an adhesive or a welding process or to use the effect produced by ultrasonic waves etc. The collar 5 may also consist of metal and be welded to the shaft, it also being possible to form the collar 5 in one piece with the shaft 4.

1. A drilling tool for a surgical drilling machine including a drill bit which comprises a shaft, a tip and a coupling member for establishing a rotary connection to a rotary drive for the drilling machine, wherein a surrounding proximal protective sleeve having a proximal and a distal end is mounted on the shaft and a distal protective sleeve is resiliently insertable into said proximal protective sleeve and surrounds the drill bit between the distal end and the tip of the drill bit over at least a portion of the length thereof, wherein the proximal protective sleeve comprises a rotation preventing means which prevents rotation of the proximal protective sleeve relative to the drilling machine when a drill bit is inserted in the drilling machine.

2. A drilling tool in accordance with claim 1, wherein the distal protective sleeve when in its extended state masks the drill bit to beyond the tip thereof.

3. A drilling tool in accordance with claim 1, wherein the proximal protective sleeve is mounted on the shaft of the drill bit such as to be rotatable about the longitudinal axis thereof.

4. A drilling tool in accordance with claim 3, wherein the proximal protective sleeve and the shaft of the drill bit comprise mutually facing open peripheral grooves in which there engages at least one common bearing element.

5. A drilling tool in accordance with claim 4, wherein the bearing element is elastically deformable and snaps resiliently into one of the two peripheral grooves when the proximal protective sleeve is displaced axially relative to the shaft.

6. A drilling tool in accordance with claim 5, wherein one peripheral groove is very shallow whilst the depth of the other peripheral groove is greater.

7. A drilling tool in accordance with claim 5, wherein the bearing element is a ring.

8. A drilling tool in accordance with claim 1, wherein the rotation preventing means is formed by a radial projection of the proximal protective sleeve which engages in a recess in the drilling machine.

9. A drilling tool in accordance with claim 1, wherein the proximal protective sleeve is fixable in the drilling machine in the axial direction.

10. A drilling tool in accordance with claim 9, wherein the proximal protective sleeve comprises at least one recess into which a locking projection of the drilling machine is insertable.

11. A drilling tool in accordance with claim 9, wherein the proximal protective sleeve carries a stop member with the aid of which it prevents displacement of the drill bit in the distal direction.

12. A drilling tool in accordance with claim 11, wherein the coupling member engages axially in form-locking manner in a coupling seating in the drilling machine.

13. A drilling tool in accordance with claim 1, wherein the shaft of the drill bit is provided with a collar which has an edge that adjoins a step of the proximal protective sleeve and comprises a peripheral groove for accommodating an O-ring, wherein said O-ring engages in an opposed peripheral groove in the proximal protective sleeve, the end face of said collar being provided with a drive means which is insertable in the form of a coupling member into a driver opening in the rotary drive for the drilling machine.

14. A drilling tool in accordance with claim 1, wherein the shaft is surrounded by a helical spring which is supported at one end on the shaft and on the distal protective sleeve at the other.

15. A drilling tool in accordance with claim 14, wherein, at at least one end thereof, the helical spring comprises an end winding which extends transversely relative to the longitudinal axis thereof and serves to support the spring on a support surface of the shaft or the distal protective sleeve.

16. A drilling tool in accordance with claim 1, wherein the distal protective sleeve is not rotatable with respect to the proximal protective sleeve about the longitudinal axis thereof.

17. A drilling tool in accordance with claim 16, wherein the distal protective sleeve is of non-round cross-section and dips into a complementary opening in the proximal protective sleeve.

18. A drilling tool in accordance with claim 1, wherein the distal protective sleeve carries a depth scale.

19. A drilling tool in accordance with claim 1, wherein there is arranged on the distal protective sleeve a slip ring which is retained thereon by friction and is displaceable in the axial direction by overcoming the frictional force.
